(19) United States
(12) Patent Application Publication
(10) Pub. No.: US 2024/0408278 A1
(43) Pub. Date: Dec. 12, 2024

(54) RAPID FORMING ARTIFICIAL BLOOD VESSEL AND PREPARATION METHOD

(71) Applicant: Chinese Academy of Medical Sciences Institute of Biomedical Engineering, Tianjin (CN)

(72) Inventors: Pengyu Huang, Tianjin (CN); Bo Deng, Tianjin (CN); Yue Ma, Tianjin (CN)

(21) Appl. No.: 18/812,148

(22) Filed: Aug. 22, 2024

(30) Foreign Application Priority Data
Dec. 15, 2023 (CN) 202311722601.9

(51) Int. Cl. A61L 27/50; A61L 27/26; A61L 27/38; C08L 5/04; C08L 71/02; C08L 89/06

(52) U.S. Cl. CPC A61L 27/507; A61L 27/26; A61L 27/3808; C08L 5/04; C08L 71/02; C08L 89/06

(57) ABSTRACT

The present invention discloses a preparation method of a rapid forming artificial blood vessel, comprising the following steps: under the condition of avoiding light, filling an annular cavity composed of an outer tube and an inner tube of a coaxial needle with a double-crosslinking photosensitive hydrogel and refrigerating at 4° C. for 8-10 minutes; filling the inner tube of the coaxial needle with CaCl₂ aqueous solution; adjusting the temperature of the coaxial needle and a printing platform; under the action of ultraviolet light, adjusting extrusion speed of the outer tube and extrusion speed of the inner tube of the coaxial needle; printing extrudates in a sterile petri dish to obtain a rapid forming artificial blood vessel; and transferring the rapid forming artificial blood vessel to the sterile petri dish containing an ECM for culturing.

### TECHNICAL FIELD

The present invention relates to a double-crosslinking photosensitive hydrogel and a rapid forming artificial blood vessel, which belongs to the field of biomaterial.

### BACKGROUND

The human body has an extremely rich and complex multistage vascularization network that performs important functions such as oxygen and nutrient supply, waste removal and signal transmission in the maintenance and operation process of organs. At present, most man-made large-scale artificial organs (such as heart, liver and kidney) lack artificial blood vessels that can be used to connect recipient blood vessels and transport nutrients for artificial organs. When a transplant grows to a certain extent, cell apoptosis and tissue necrosis will occur due to the lack of effective oxygen and nutrition in the tissue. At the same time, the large-scale rapid preparation of artificial blood vessels containing cells is also a major problem at present. Therefore, artificial blood vessels prepared by 3D bioprinting, etc. have attracted wide attention from researchers in recent years.

At present, the most common method to achieve vascularization of tissues and organs is to mix endothelial cells (ECs) and functional cells (liver, kidney and myocardial cells) in hydrogels with good biocompatibility and mechanical properties, such as ECM, gelatin, etc. and finally perform self-assembly through the proliferation and migration of the ECs in the hydrogels to form a capillary network. However, capillaries formed entirely by self-assembly have low integration efficiency in a recipient body, cannot be quickly connected with main blood vessels in the recipient body, and cannot achieve the effect of salvage treatment for some patients with acute organ failure. Therefore, there is an urgent need for an artificial blood vessel capable of transporting nutrients to artificial organs and connecting directly with the blood vessels of a recipient.

### SUMMARY

A purpose of the present invention is to overcome the shortcomings of the prior art to provide a double-crosslinking photosensitive hydrogel.

A second purpose of the present invention is to provide a rapid forming artificial blood vessel.

A third purpose of the present invention is to provide a preparation method of a rapid forming artificial blood vessel.

A technical solution of the present invention is summarized as follows:

A double-crosslinking photosensitive hydrogel is prepared by the following method:
1) taking 8 g-12 g of gelatin methacryloyl, 0.4 g-0.8 g of sodium alginate, 0.04 g-0.08 g of polyethylene oxide with an average molecular weight of 200000-400000, 0.2 g-0.4 g of lithium phenyl(2,4,6-trimethylbenzoyl)phosphinate, 1-5×10⁸ human umbilical vein endothelial cells and the balance of phosphate buffer solution in proportion, with a total of 100 ml;
2) under the condition of avoiding light, evenly mixing the gelatin methacryloyl, the sodium alginate, the polyethylene oxide with an average molecular weight of 200000-400000, the phosphate buffer solution, the human umbilical vein endothelial cells and the lithium phenyl(2,4,6-trimethylbenzoyl)phosphinate in sequence to obtain the double-crosslinking photosensitive hydrogel.

A preparation method of a rapid forming artificial blood vessel comprises the following steps:
under the condition of avoiding light, filling an annular cavity composed of an outer tube and an inner tube of a coaxial needle with the double-crosslinking photosensitive hydrogel and refrigerating at 4° C. for 8-10 minutes; filling the inner tube of the coaxial needle with CaCl₂ aqueous solution with a mass concentration of 1%-3%; adjusting the temperature of the coaxial needle to 10-15° C., and adjusting the temperature of a printing platform to 5-10° C.; under the action of 405 nm ultraviolet light, adjusting extrusion speed of the outer tube of the coaxial needle to 2-3 mm³/s, and adjusting extrusion speed of the inner tube of the coaxial needle to 5 mm³/s; printing extrudates in a sterile petri dish to obtain a rapid forming artificial blood vessel; and transferring the rapid forming artificial blood vessel to the sterile petri dish containing an ECM for culturing.

A rapid forming artificial blood vessel prepared by the preparation method is provided.

The present invention has the following advantages:

The double-crosslinking photosensitive hydrogel of the present invention can be formed rapidly under the action of double curing of CaCl₂ solution and 405 nm ultraviolet light, maintains the shape of a blood vessel for a long time, and has good biocompatibility and 3D printing performance.

The rapid forming artificial blood vessel of the present invention can be produced massively in batch in vitro by 3D bioprinting technology, and exhibits the functions of biomacromolecules and glucose transportation in vitro. In in-vivo models, the rapid forming artificial blood vessel can be surgically anastomosed with rat veins to achieve systemic blood circulation through the artificial blood vessel.

### DESCRIPTION OF DRAWINGS

FIG. 1 shows a rapid forming artificial blood vessel;

FIG. 2 shows the in vitro transportation of macromolecular substances of the rapid forming artificial blood vessel;

FIG. 3 shows the in vitro glucose transportation of a rapid forming artificial blood vessel;

FIG. 4 shows the venous anastomosis of a rapid forming artificial blood vessel in a rat.

### DETAILED DESCRIPTION

All raw materials are commercially available.

The present invention is further illustrated below through specific embodiments.

### Embodiments 1-3 Double-Crosslinking Photosensitive Hydrogel

The raw material formulas of three embodiments are shown in Table 1, with 100 ml as an example.

| | Embodiment | | Embodiment | | Embodiment | |
|---|---|---|---|---|---|---|
| | 1 | | 2 | | 3 | |
| Gelatin methacryloyl | 10 | g | 8 | g | 12 | g |
| Sodium alginate | 0.6 | g | 0.8 | g | 0.4 | g |
| Polyethylene oxide | Average | | Average | | Average | |
| | molecular | | molecular | | molecular | |
| | weight of | | weight of | | weight of | |
| | 300000 | | 200000 | | 400000 | |
| | 0.06 g | | 0.04 g | | 0.08 g | |
| Lithium | 0.3 | g | 0.4 | g | 0.2 | g |
| phenyl(2,4,6- | | | | | | |
| trimethylbenzoyl)phosphinate | | | | | | |

| | | | |
|---|---|---|---|
| Human umbilical vein | 3 × 10⁸ | 1 × 10⁸ | 5 × 10⁸ |
| endothelial cells | | | |
| Phosphate buffer solution | Balance | Balance | Balance |

Any volume can be prepared by scale.

Under the condition of avoiding light, according to Table 1, the gelatin methacryloyl, the sodium alginate, the polyethylene oxide with an average molecular weight of 200000-400000, the phosphate buffer solution, the human umbilical vein endothelial cells and the lithium phenyl(2,4,6-trimethylbenzoyl)phosphinate are evenly mixed in sequence to obtain the double-crosslinking photosensitive hydrogels of embodiment 1, embodiment 2 and embodiment 3 respectively.

### Embodiment 4

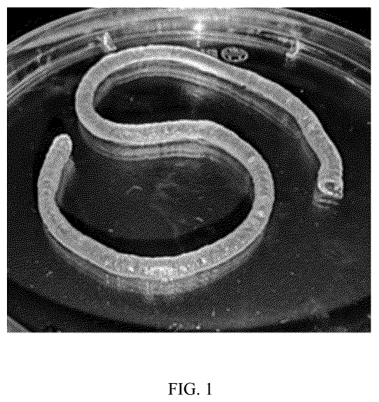

A preparation method of a rapid forming artificial blood vessel comprises the following steps:
under the condition of avoiding light, filling an annular cavity composed of an outer tube and an inner tube of a coaxial needle (two commercial needles are 13G and 8G respectively) with the double-crosslinking photosensitive hydrogel of embodiment 1, and refrigerating at 4° C. for 9 minutes; filling the inner tube of the coaxial needle with CaCl₂ aqueous solution with a mass concentration of 2%; adjusting the temperature of the coaxial needle to 13° C., and adjusting the temperature of a printing platform to 8° C.; under the action of 405 nm ultraviolet light, adjusting extrusion speed of the outer tube of the coaxial needle to 2.5 mm³/s, and adjusting extrusion speed of the inner tube of the coaxial needle to 5 mm³/s; printing extrudates in a sterile petri dish to obtain a rapid forming artificial blood vessel, as shown in FIG. 1; and transferring the rapid forming artificial blood vessel to the sterile petri dish containing an ECM for culturing.

### Embodiment 5

A preparation method of a rapid forming artificial blood vessel comprises the following steps:
under the condition of avoiding light, filling an annular cavity composed of an outer tube and an inner tube of a coaxial needle (two commercial needles are 13G and 8G respectively) with the double-crosslinking photosensitive hydrogel of embodiment 2, and refrigerating at 4° C. for 8 minutes; filling the inner tube of the coaxial needle with CaCl₂ aqueous solution with a mass concentration of 1%; adjusting the temperature of the coaxial needle to 10° C., and adjusting the temperature of a printing platform to 5° C.; under the action of 405 nm ultraviolet light, adjusting extrusion speed of the outer tube of the coaxial needle to 2 mm³/s, and adjusting extrusion speed of the inner tube of the coaxial needle to 5 mm³/s; printing extrudates in a sterile petri dish to obtain a rapid forming artificial blood vessel (with the shape similar to that of the artificial blood vessel prepared in embodiment 4); and transferring the rapid forming artificial blood vessel to the sterile petri dish containing an ECM for culturing.

### Embodiment 6

A preparation method of a rapid forming artificial blood vessel comprises the following steps:
under the condition of avoiding light, filling an annular cavity composed of an outer tube and an inner tube of a coaxial needle (two commercial needles are 13G and 8G respectively) with the double-crosslinking photosensitive hydrogel of embodiment 3, and refrigerating at 4° C. for 10 minutes; filling the inner tube of the coaxial needle with CaCl₂ aqueous solution with a mass concentration of 3%; adjusting the temperature of the coaxial needle to 15° C., and adjusting the temperature of a printing platform to 10° C.; under the action of 405 nm ultraviolet light, adjusting extrusion speed of the outer tube of the coaxial needle to 3 mm³/s, and adjusting extrusion speed of the inner tube of the coaxial needle to 5 mm³/s; printing extrudates in a sterile petri dish to obtain a rapid forming artificial blood vessel (with the shape similar to that of the artificial blood vessel prepared in embodiment 4); and transferring the rapid forming artificial blood vessel to the sterile petri dish containing an ECM for culturing.

The diameters of two commercial needles of the coaxial needle can be adjusted to obtain a rapid forming artificial blood vessel with different diameters and different thicknesses.

### Embodiment 7

### Detection:

### (1) Biomacromolecule Diffusion

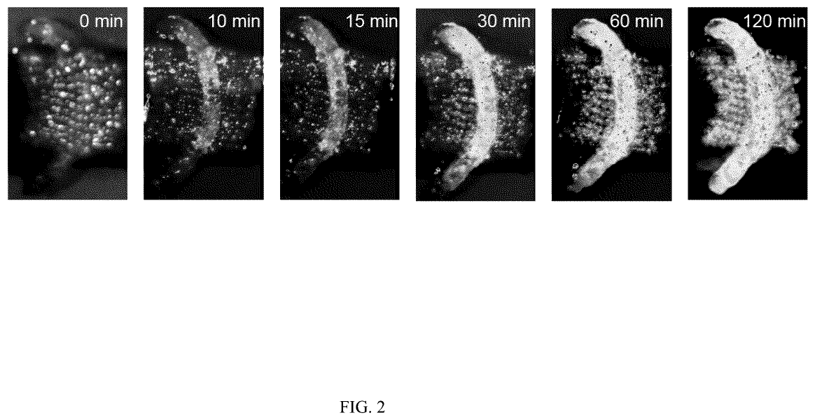

In vitro simulated perfusion is performed for the rapid forming artificial blood vessel prepared in embodiment 4 by using the phosphate buffer solution containing CY7 fluorescent dye (0.1 mg/ml, APE×BIO). The perfusion speed is adjusted to 6 ml/min. Fluorescence intensity in different parts of a printed body through which the blood vessel flows is monitored by Maestro imaging system (CRi Maestro) at 0 min, 10 min, 15 min, 30 min, 60 min and 120 min after the perfusion begins, as shown in FIG. 2.

### (2) Glucose Accumulation

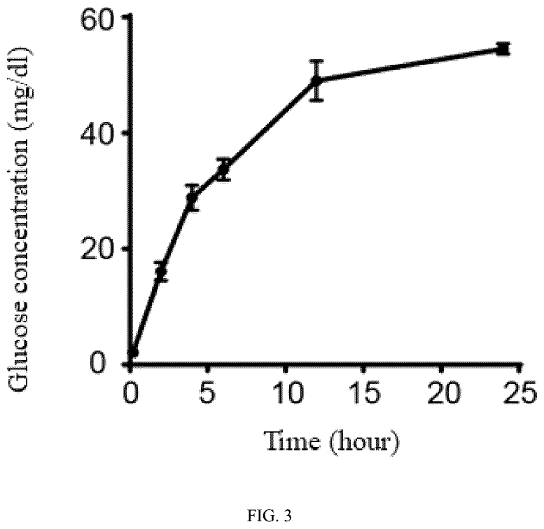

The rapid forming artificial blood vessel prepared in embodiment 4 is placed in a 6 cm petri dish containing phosphate buffer solution. Both ends of the blood vessels are placed outside the petri dish. In vitro perfusion is performed for the blood vessel by using the phosphate buffer solution containing glucose (100 mg/dl), and the glucose concentration of the phosphate buffer solution in the petri dish is detected at different times after the perfusion begins, as shown in FIG. 3.

### (3) Venous Anastomosis of a Rapid Forming Artificial Blood Vessel in a Rat

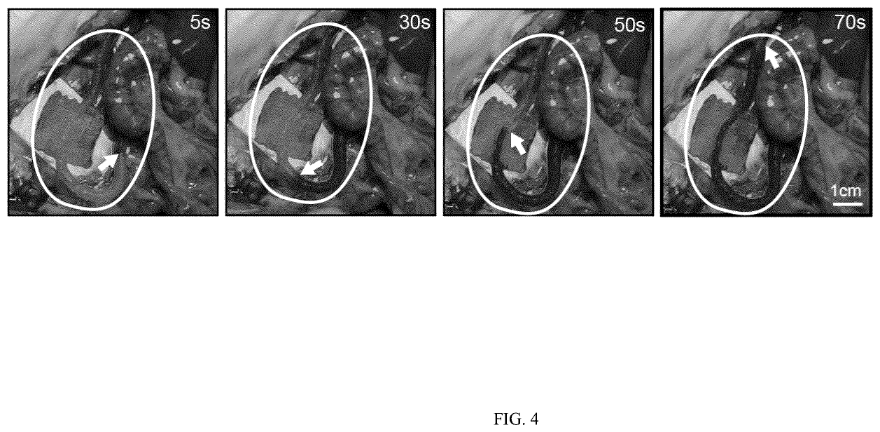

An SD rat aged 10-12 weeks (an SD rat aged 12 weeks is used in this experiment) is selected for an anastomosis experiment of the artificial blood vessel. The rat is rapidly anesthetized with 3% isoflurane before the operation. During the operation, hemostatic clips are used for clamping the ends of the inferior vena cava and the hepatic portal vein of the rat to temporarily stop the blood flow. A cuff is inserted respectively into both ends of the inferior vena cava and the hepatic portal vein and fixed with 6-0 surgical sutures. Both ends of the rapid forming artificial blood vessel prepared in embodiment 4 are inserted into the cuffs, and then the hemostatic clips are removed to establish hemoperfusion, as shown in FIG. 4.

The experiment shows that the experimental results of the rapid forming artificial blood vessels prepared in embodiments 5 and 6 are similar to those of the rapid forming artificial blood vessel prepared in embodiment 4 in the experiments of biomacromolecule diffusion, glucose accumulation and venous anastomosis in the rat.

What is claimed is:

1. A double-crosslinking photosensitive hydrogel, prepared by the following method:
1) taking 8 g-12 g of gelatin methacryloyl, 0.4 g-0.8 g of sodium alginate, 0.04 g-0.08 g of polyethylene oxide with an average molecular weight of 200000-400000, 0.2 g-0.4 g of lithium phenyl(2,4,6-trimethylbenzoyl)phosphinate, 1-5×10⁸ human umbilical vein endothelial cells and the balance of phosphate buffer solution in proportion, with a total of 100 ml;
2) under the condition of avoiding light, evenly mixing the gelatin methacryloyl, the sodium alginate, the polyethylene oxide with an average molecular weight of 200000-400000, the phosphate buffer solution, the human umbilical vein endothelial cells and the lithium phenyl(2,4,6-trimethylbenzoyl)phosphinate in sequence to obtain the double-crosslinking photosensitive hydrogel.

2. A preparation method of a rapid forming artificial blood vessel, comprising the following steps:
under the condition of avoiding light, filling an annular cavity composed of an outer tube and an inner tube of a coaxial needle with the double-crosslinking photosensitive hydrogel according to claim 1 and refrigerating at 4° C. for 8-10 minutes; filling the inner tube of the coaxial needle with CaCl₂ aqueous solution with a mass concentration of 1%-3%; adjusting the temperature of the coaxial needle to 10-15° C., and adjusting the temperature of a printing platform to 5-10° C.; under the action of 405 nm ultraviolet light, adjusting extrusion speed of the outer tube of the coaxial needle to 2-3 mm³/s, and adjusting extrusion speed of the inner tube of the coaxial needle to 5 mm³/s; printing extrudates in a sterile petri dish to obtain a rapid forming artificial blood vessel; and transferring the rapid forming artificial blood vessel to the sterile petri dish containing an ECM for culturing.

3. A rapid forming artificial blood vessel prepared by the preparation method according to claim 2.
